(19) United States
(12) Patent Application Publication
(10) Pub. No.: US 2011/0306077 A1
(43) Pub. Date: Dec. 15, 2011

(54) Methods for the detection of alternative cellular energy (ACE) pigments and for monitoring of the ACE pathway in the diagnosis and therapy of diseases

(76) Inventor: William John Martin, South Pasadena, CA (US)

(21) Appl. No.: 12/802,763

(22) Filed: Jun. 13, 2010

(51) Int. Cl. C12Q 1/02

(52) U.S. Cl. 435/29

(57) ABSTRACT

Ultraviolet light reactive alternative cellular energy pigments (ACE-pigments) are produced in response to certain chronic viral infections, metabolic disorders and degenerative illnesses. The pigments will display differing responsiveness to direct ultraviolet (UV) light illumination in the presence and in the absence of certain triggering dyes, including neutral red. The type of response appears to reflect varying energy levels of the ACE pigments from being essentially uncharged (fluorescent but only if triggering dye is added); to partially charged (fluorescent even in the absence of a triggering dye) to more fully charged (non-fluorescent either in the presence or absence of the triggering dye). Methods are described for assessing the status of ACE pigments within localized skin and deeper lesions, and within the overall body of a human or animal subject. The methods can be used to help monitor the efficacy of therapeutic measures aimed at enhancing the ACE pathway in those in whom a deficiency in the pathway can be shown to exist.

Co-pending with the following related patent applications:

Methods for Detection of Ultraviolet Light Reactive Alternative Cellular Energy Pigments (ACE-pigments) William John Martin Filed Dec. 24, 2007. Number 20090163831 Method of Assessing and of Activating the Alternative Cellular Energy (ACE) Pathway in the Therapy of Diseases. William John Martin Filed Jan. 17, 2008. Number 20090181467

Enerceutical activation of the alternative cellular energy (ACE) pathway in therapy of diseases. William John Martin Filed Feb. 11, 2008 Number 20090202442 Enerceutical mediated activation of the Alternative Cellular Energy (ACE) pathway in the therapy of diseases. William John Martin Filed May 8, 2008 Number 20090280193

Moringa Oil Mediated Activation of the Alternative Cellular Energy Pathway in the Therapy of Diseases. William John Martin Field Feb. 24, 2010.

Enerceutical Mediated Activation of the Alternative Cellular Energy (ACE) Pathway in the Therapy of Diseases. William John Martin Filed Jun. 9, 2010

Previously Submitted but Now Abandoned Patent Applications

Ser. No. 10/044,683. Therapy of stealth virus associated cancers and other conditions using light. William John Martin. (Abandoned)

Ser. No. 10/047,313. Therapy of stealth virus associated cancers and other conditions using medium chain triglycerides. William John Martin. (Abandoned)

Ser. No. 10/050,232. Diagnosing and monitoring the therapy of stealth virus infections based on the detection of auto-fluorescent material in hair. William John Martin. (Abandoned)

Ser. No. 10/058,480. Therapy of stealth virus associated cancers and other conditions using magnetic energy. William John Martin. (Abandoned)

Ser. No. 10/164,258 Energy supportive therapy of stealth virus associated diseases. William John Martin. (Abandoned)

Ser. No. 10/174,466 Sound therapy of stealth virus associated diseases. William John Martin. (Abandoned)

Ser. No. 10/192,936 ACE-Pigments and humic acids as energy sources. William John Martin. (Abandoned)

Submitted: 10/______ Methods for Collection of Alternative Cellular Energy Pigments (ACE-pigments). William John Martin. (Abandoned)

Submitted: 10/______ Methods for Elimination of Toxic Alternative Cellular Energy Pigments (ACE-pigments) and for Their Replacement Using Activated Humates, including Humic and Fulvic Acids. William John Martin. (Abandoned)

United States Patents (Awarded)

U.S. Pat. No. 5,985,546 Stealth virus detection in the chronic fatigue syndrome. William John Martin

U.S. Pat. No. 5,891,468 Stealth virus detection in the chronic fatigue syndrome. William John Martin

U.S. Pat. No. 5,753,488 Isolated stealth viruses and related vaccines. William John Martin

U.S. Pat. No. 5,703,221 Stealth virus nucleic acids and related methods. William John Martin

PCT (Patent Cooperation Treaty)

WO 92/20797 Stealth virus detection in the chronic fatigue syndrome. William John Martin

WO 99/34019 Stealth virus nucleic acids and related methods. William John Martin

WO 99/60101 Stealth viruses and related vaccines. William John Martin

### REFERENCES TO PUBLISHED ARTICLES

Alternative Cellular Energy Pigments (ACE-pigments):
1 Martin W J. Alternative cellular energy pigments mistaken for parasitic skin infestations. Exp. Mol. Path 78: 212-214, 2005.
2 Martin W J. Alternative cellular energy pigments from bacteria of stealth virus infected individuals. Exp. Mol. Path 78: 217-217, 2005.
3 Martin W J. Progressive Medicine. Exp Mol Path 78: 218-220, 2005.
4 Martin W J, Stoneburner J. Symptomatic relief of herpetic skin lesions utilizing an energy based approach to healing. Exp. Mol. Path 78: 131-4, 2005.
5 Martin W J. Etheric Biology. Exp Mol Path 78: 221-227, 2005.
6 Martin W J. Stealth Virus Culture Pigments: A Potential Source of Cellular Energy. Exp. Mol. Pathol. 74: 210-223, 2003.
7 Martin W J. Complex intracellular inclusions in the brain of a child with a stealth virus encephalopathy. Exp. Mol. Pathol. 74: 179-209, 2003.
8 Martin W J. Photons and phonons: Theoretical aspects of biophysics and potential therapeutic applications. Proceeding of Neural Therapy Workshop on Sound and Light Therapy, Seattle, Wash., Feb. 21-23, 2003.

Stealth Adapted Viruses
1 Martin W J Chronic fatigue syndrome among physicians. A potential result of occupational exposure to stealth viruses. Explore 2001; 10: 7-10.
2 Martin W J. Stealth Viruses. Explore 2001; 10: 17-19.
3 Durie G M, Collins R. Martin W J. Positive stealth virus cultures in multiple myeloma. A possible explanation for neuropsychiatric co-morbidity. Presented at the Am. Soc. Hematology annual meeting October 2000.
4 Martin W J. Chemokine receptor-related genetic sequences in an African green monkey simian cytomegalovirus-derived stealth virus. Exp Mol Pathol. 2000; 69:10-6.
5 Martin W J., Anderson D. Stealth virus epidemic in the Mohave Valley: severe vacuolating encephalopathy in a child presenting with a behavioral disorder. Exp Mol Pathol. 1999; 66:19-30.
6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.
7 Martin W J. Bacteria-related sequences in a simian cytomegalovirus-derived stealth virus culture. Exp Mol Pathol. 1999; 66:8-14.
8 Martin W J. Stealth adaptation of an African green monkey simian cytomegalovirus. Exp Mol Pathol. 1999; 66:3-7.
9 Martin W J. Cellular sequences in stealth viruses. Pathobiology 1998; 66:53-8.
10 Martin W J. Detection of RNA sequences in cultures of a stealth virus isolated from the cerebrospinal fluid of a health care worker with chronic fatigue syndrome. Case report. Pathobiology. 1997; 65:57-60.
11 Martin W J., Anderson D. Stealth virus epidemic in the Mohave Valley. I. Initial report of virus isolation. Pathobiology. 1997; 65:51-6.
12 Martin W J. Simian cytomegalovirus-related stealth virus isolated from the cerebrospinal fluid of a patient with bipolar psychosis and acute encephalopathy. Pathobiology. 1996; 64:64-6.
13 Martin W J. Stealth viral encephalopathy: report of a fatal case complicated by cerebral vasculitis. Pathobiology. 1996; 64:59-63.
14 Martin W J. Genetic instability and fragmentation of a stealth viral genome. Pathobiology. 1996; 64:9-17.
15 Martin W J. Severe stealth virus encephalopathy following chronic-fatigue-syndrome-like illness: clinical and histopathological features. Pathobiology. 1996; 64:1-8.
16 Martin W J. Stealth virus isolated from an autistic child. J Autism Dev Disord. 1995; 25:223-4.
17 Gollard R P, Mayr A., Rice D A, Martin W J. Herpes virus-related sequences in salivary gland tumors. J Exp Clin Cancer Res., 1996; 15: 1-4.
18 Martin W J., Glass R T. Acute encephalopathy induced in cats with a stealth virus isolated from a patient with chronic fatigue syndrome. Pathobiology. 1995; 63:115-8.
19 Martin W J, et al. African green monkey origin of the atypical cytopathic ‘stealth virus’ isolated from a patient with chronic fatigue syndrome. Clin Diag Virol 1995: 4: 93-103.
20 Martin W J. Stealth viruses as neuropathogens. CAP Today. 1994; 8: 67-70.
21 Martin W J. et al. Cytomegalovirus-related sequence in an atypical cytopathic virus repeatedly isolated from a patient with chronic fatigue syndrome. Am J Pathol. 1994; 145: 440-51.
22 Martin W J. Activation of the alternative cellular energy (ACE) pathway as natural therapy for patients with autism. Submitted Mar. 27, 2008 to J. Autism and Developmental Disorders.
23. Martin W J. Activation of the alternative cellular energy (ACE) pathway as natural therapy for herpes simplex and herpes zoster virus infections. Submitted Mar. 27, 2008 to J. Infectious Diseases and subsequently to J. Complimentary and Alternative Medicine (Not accepted).
24. Activation of the Alternative Cellular Energy (ACE) Pathway as Natural Therapy for Patients with Autism. Submitted originally to J. autism and Developmental Disorder, and in a revised and expanded form to Proceedings of the National Academy of Sciences and next to Autism. Not accepted by these journals.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

As described in the co-pending patent applications, alternative cellular energy pigments (ACE-pigments) provide a source of cellular energy, which is different from that provided through the oxidative metabolism of foods, or in the case of plants and certain bacteria, through the process of photosynthesis. They appear to comprise various types of naturally synthesized organometalic complexes with energy capturing, storing and transmitting capacities and to function in all living creatures. Using the analogy of miniature batteries, the energy levels of ACE pigments in humans and animals have been classified as being essentially “uncharged” when they will fluoresce when exposed to ultraviolet (UV) light, but only if first mixed with a suitable triggering dye, such as neutral red. Upon “partial charging,” the ACE pigments will now also fluoresce when directly illuminated with a suitable UV light, even in the absence of a triggering dye such as neutral red. When “fully charged,” ACE pigments will not receive additional energy input and will no longer fluoresce when UV illuminated, with or without the presence of neutral red dye. ACE pigments can also respond to other energy sources, including electrostatic, magnetic and sound.

ACE pigment particles will also demonstrate other properties consistent with their energy converting (transducing) capacity. These include non-Brownian movement, as seen microscopically, especially with fluorescing neutral red or acridine orange stained particles; electron donating and accepting activity; electrostatic and occasionally magnetic properties; ability to generate gas bubbles from water; and ability to suppress the cytopathic (cell damaging effects) of virus infections. Excessive stimulation of ACE pigments within tissue culture medium can, by itself, lead to cell damage, especially with single cell microorganisms.

Activating ACE pigments in humans and animals with a deficiency of the ACE pathway is a therapeutic procedure that can seemingly enhance normal cellular functions and promote the recovery from various infectious, metabolic and degenerative diseases. Activation can be achieved by several methods described in the cited co-pending patent applications, which are incorporated herein by reference.

This patent application discloses various methods that are useful in the initial assessment of, and in the therapeutic monitoring of, the energy level of the ACE pathway in humans and in animals subjects. The assessment and monitoring can be applied to localized skin and other lesions and also to the overall body (systemic) pathway acting within the subject. The present application reports on some improvements in the methods that were disclosed in prior patent applications.

### BRIEF SUMMARY OF THE INVENTION

The major changes are i) the use neutral red dye absorbed onto a suitable filter paper for the testing of bodily fluids and of materials collected from localized skin lesions for the presence of uncharged ACE pigments (as shown by UV fluorescence of the dried spot of neutral red to which the bodily material is added; and ii) the routine use of ethyl alcohol and of water to provide a positive and a negative control, respectively for the neutral red—ACE pigments fluorescence testing. These changes will encourage the widespread adoption of the procedure for self-testing and for parental testing of children. ACE test kits can also include the use of glass filters that selectively transmit UV light into a darkened container in which the fluorescence can be more easily detected and/or photographed. The source of UV lighting can be either a mercury vapor lamp or a UV Light Emitting Diode (LED).

### BRIEF DESCRIPTION OF THE DRAWINGS

Not Applicable and none included

### DETAILED DESCRIPTION OF THE INVENTION

Efforts have been underway to provide a simple procedure for individuals to assess the energy level of their own ACE pathway or that of their children. This assessment can be useful in determining the need for, and the effectiveness of, various measures aimed at activating ACE pigments. The basic procedures have been addressed in the co-pending patent applications. They comprise observing various skin lesions, uninvolved areas of skin, hair, bodily fluids and the open mouth for yellow to orange to red fluorescence under direct illumination with an UV light. If no direct fluorescence is observed, materials from the body are typically then collected and mixed with a small quantity of a neutral red dye (approximately 0.1-0.5 mg/ml) and reexamined for fluorescence. In patients without specific skin lesions for ACE pigment testing, a preference is generally given to either sampling of saliva or of perspiration. Although not as commonly practiced because of FDA requirements, neutral red dye can also be directly applied to a localized skin lesion or to other areas of the body and the site examined for UV fluorescence. The addition of neutral red dye is not required to elicit direct UV fluorescence of partially charged ACE pigments, either remaining on the body or freshly removed from the body. Uncharged, partially charged but not fully charged ACE pigments will yield fluorescence when in combination with neutral red dye and illuminated with a UV light. Uncharged and partially charged ACE pigments can also markedly alter the fluorescence emitted by certain fluorescence dyes, notable acridine orange, proflavine and Stains-All.

In promoting ACE pigment—neutral red testing for home use, I needed to feel confident that individuals would fully understand the difference between fluorescence and reflected light. I also knew that some individuals were observing spurious fluorescence because of the materials they were using to collect bodily samples. Neutral red will fluoresce when dissolved in neutral red but not when dissolved in water (except at very high pH). I can, therefore, easily demonstrate neutral red fluorescence using ethyl alcohol (or other alcohols, including methanol, propanol, etc.) Individuals can now quickly grasp the nature of fluorescence. Another major advance has been providing neutral red dried onto suitable absorbent material. I knew that it was important to exclude some types of paper and cloth products on which neutral red would show varying degrees of direct fluorescence. This is probably due to chemical additives to these products. Consistent with this suggestion, neutral red will not show immediate or delayed UV fluorescence when it is applied to laboratory filters, coffee filters and even toilet paper, which are presumably devoid of toxic chemicals. Dried spots of neutral red will fluoresce, however, if subsequently moistened with bodily fluids from a patient with a deficiency of the ACE pathway. In a typical test, I will align three spots of neutral red dye onto a sheet of laboratory filter paper. One drop will be moistened with water (negative control), one with ethyl alcohol (positive control) and one with the patient's material (test sample). A separate drop of the patient sample will be placed elsewhere on the filter to test for direct UV fluorescence. The UV lighting can be either a tubular or spiral UV mercury light bulb, or a UV Light Emitting Diode (LED). A suitable example is the 15 watt ProLume ultra compact mini-spiral mercury vapor black light obtained from Halco Light Technologies, Norcross Ga. It has a maximum emission at 365 nm. A glass plate that allows the transmission of UV light, but reflects visible light can also be positioned between the UV light source and neutral red spots. Suitable examples are Kodak Wratten 18A filter, Schneider Optics, New York, filter code number 403; or Rolyn Optics Company, Covina Calif., catalogue number 65.1030. The glass plate acts as a mirror and clearly shows whether the neutral red spots are fluorescing. A camera can be positioned to photograph the spots or their reflection in the glass plate. Photographs are useful as an electronic transferrable, permanent record of the fluorescence, as well as providing information on the spectrum of colors that can be observed from bright yellow through orange and purple to reddish coloring.

The test sample can be material collected from a specific skin lesion, e.g. a possible herpes eruption. Samples of bodily fluids such as perspiration, saliva, urine or serum/plasma can be similarly tested. The available amounts of perspiration can be increased by heat, exercise and refraining from bathing. Niacin and some herbal products, such as Sweet Sweat,™ also promote sweating.

The testing of materials collected from the body for ACE pigments can provide a useful indication for whether or not to proceed with ACE pathway activation therapy. With specific skin lesions, examination of material obtained from the lesion can also be helpful in distinguishing between chronic conditions, such as recurrent herpes in which ACE pigments are expected to be present and easily detectable, from more acute lesions, such traumatic or short-term bacterial infections, in which ACE pigments are unlikely to have accumulated. Variable findings have been seen in preliminary studies with other skin conditions, including psoriasis, eczema and solar keratosis. Comparable studies in many of these conditions can be performed by directly applying neutral red dye to the skin lesion. The U.S. Food and Drug Administration (FDA) has expressed its concern that neutral red could possibly cause herpes viruses to mutate if added to an active herpes skin lesion. The same concern does not exist when the neutral red staining is applied to intact skin or even to the mucus membrane of the mouth. The external testing of neutral red inducible fluorescence as described above is still preferable for cosmetic reasons and for the remote possibility of skin sensitization, especially when repeated testing is anticipated. Moreover, FDA considers any direct application of neutral red to the body as the use of a drug with would face more regulatory hurdles than its approval as a stain to be used without direct contact with the body.

The direct UV light testing of skin lesions, normal appearing skin and, particularly, the oral cavity, without using neutral red dye, is particularly useful in determining whether an ACE activation therapeutic procedure is having its desired effect. As disclosed in earlier co-pending patent applications, certain types of skin lesions contain uncharged ACE pigments, defined as having removable material that will fluoresce under UV light when mixed with neutral red dye. A goal in treating such lesions is to place fluorescing material in close proximity to the lesion to allow for an energy transfer into the lesion to occur. The energy transfer causes the underlying skin lesion itself, and sometimes the surrounding skin, to fluoresce when illuminated with UV light, even though no neutral red is present in the lesion. The fluorescing material that is placed in close proximity to the active skin lesion can be either i) portion of the material removed from the lesion, ii) ACE pigments collected elsewhere from the patient, e.g., saliva or perspiration, or iii) enerceuticals,™ including a fresh alcohol solution of neutral red. Periodic examination of the underlying skin lesion for direct UV inducible fluorescence is, therefore, useful as an indication of energy transfer. Moreover, if desired the direct UV illumination of the now fluorescing skin lesion can be continued until it fades (fully charged). Oral fluorescence and florescence of distant skin lesions that may be present in such patients can also be commonly observed during the procedure. Therapy in patients without localized skin lesions, e.g. in children with autism, can be similarly monitored on the basis of induced deep skin fluorescence and oral cavity fluorescence. The detection of oral cavity fluorescence is more sensitive than skin fluorescence and can last for much longer periods. A treated autistic child with post treatment, residual skin and oral cavity fluorescence seemingly responded favorably to playing outdoors in the sunlight.

The mother of the autistic child has reported on very marked improvement in her child's behavior from the periodic activation of the child's ACE pathway. She regularly monitors her child's oral cavity for direct fluorescence and her saliva for neutral red inducible fluorescence. Moreover, her child's direct oral fluorescence has been sustainable for periods beyond a week following the ACE pathway activation procedure. Her saliva still fluoresces when mixed with neutral red. The mother's goal is for her daughter's saliva to no longer fluoresce when mixed with neutral red. A similar goal has been set for ongoing studies in another child with autism, a patient with breast cancer, two young adults with bipolar psychoses, several patients with chronic fatigue-like illness and the dog of one of the patients. The dog has strongly fluorescing saliva when it is added to dried spots of neutral red on laboratory filter paper.

Surgically removed tissues and both fine needle aspirated or exfoliative cytology specimens can be examined for intracellular and extracellular direct and neutral red inducible UV fluorescing materials. A fluorescent microscope is used for this purpose. Similarly, blood smears can be studied, with particular attention paid to neutral red stainable fluorescing granular materials within polymorphonuclear cells. The aim of these studies is again to obtain an indication of the energy level of the patient's ACE pathway. Comparable studies can similarly be performed in animal.

In all of the above studies, it is understood that substances other than ACE pigments may cause fluorescence when mixed with neutral red. For example, alcohol solutions of neutral red will fluoresce brightly. Similarly, several vitamins, tryphtophan rich proteins, porphyrins and other substances can direct fluoresce under UV illumination. Still from a practical point of view, the finding of neutral red inducible fluorescence in bodily fluids is a good presumptive argument for the probable presence of uncharged ACE pigments. Similarly, the appearance of oral fluorescence in someone undergoing enerceutical™ mediated therapy is a solid indication of a prior deficiency in their ACE pathway. A major value of the testing is that it can be easily performed by individuals within the home setting and can be regularly repeated as a means of monitoring the outcome of various therapies, even beyond the use of enerceuticals.™ The detection and the monitoring of the ACE pathway based on neutral red fluorescence can also be usefully explored in the diagnostic laboratory. Fluorescent microscopy and UV spectrophotometers are widely available in most clinical laboratories, including virus culture facilities. Clinical laboratories can also have easier access to alternative dyes that are regarded as being more toxic than neutral red and, therefore, less appropriate for home use. For example, acridine orange and Stains-All are very suitable dyes for the staining of ACE pigments within cells and virus culture fluids.

The principles, preferred embodiments and modes of operation of the present invention have been described in the foregoing specification. The diagnostic uses of the methods for assessing and of monitoring the ACE pathway, which are intended to be protected herein, are illustrative of possible applications, which can even be extended to plants. Basically, the claims relate to the entirety of methods useful in assessing the status of the ACE pathway using fluorescence and of monitoring changes in the pathway as a result of directed therapies. The claims are also intended to cover additional assays of ACE pigments that are based on their energy transducing properties, including non-Brownian motility and ability to dissociate water molecules into gas bubbles. Additional advantages and modifications will readily occur to those skilled in the art and especially upon practicing the currently described methods. Variations and changes may be made without departing from the spirit of the invention encompassed by the appended claims.

What I claim as my invention is:

1. A method for determining whether uncharged alternative cellular energy (ACE) pigments are present in a subject comprising: i) collecting bodily fluids and/or other material of the subject; ii) interacting the material with neutral red dye; and iii) examining the material-dye mixture for fluorescence using ultraviolet (UV) light illumination; such that the appearance of fluorescence of the mixture is evidence for the presence of uncharged ACE pigments in the tested material.

2. The method of claim 1 in which the neutral red dye is added directly to an area of skin, mucus membrane or a defined lesion within the skin or mucus membrane; and the site to which the neutral red dye is added is examined for fluorescence under UV illumination; such that the appearance of fluorescence, which was not observable using UV illumination prior to the addition of the neutral red dye, is evidence for the presence of uncharged ACE pigments in the site of the body being examined.

3. A method for determining whether partially charged alternative cellular energy (ACE) pigments are present in a subject comprising: i) collecting bodily fluids and/or other material of the subject; and ii) examining the material-dye mixture for fluorescence using ultraviolet (UV) light illumination; such that the appearance of fluorescence of the mixture is evidence for the presence of partially charged ACE pigments in the tested material.

4. The method of claim 3 in which an area of skin, mucus membrane or a defined lesion within the skin or mucus membrane is examined for fluorescence under UV illumination; such that the appearance of direct fluorescence is evidence for the presence of partially charged ACE pigments in the site of the body being examined.

5. The method of claim 1 in which a droplet of neutral red dye solution in water is absorbed onto suitable absorbent material, such as a laboratory filter, and allowed to dry, and that after drying the neutral red will not fluoresce by itself, but will do so if exposed to ethyl alcohol but not to water, and the dried droplet will further allow for the detection of uncharged ACE pigments, as seen by fluorescence occurring when fluids containing uncharged ACE pigments are added to the dried droplet of neutral red dye.

6. The method of claim 3 in which the appearance of partially charged ACE pigments, during or shortly after a therapeutic procedure is performed in a subject, in whom only uncharged ACE pigments could be demonstrated prior to the therapeutic procedure, provides evidence of the effectiveness of the therapeutic procedure in achieving at least partial charging of ACE pigments within the subject.

7. A kit to perform the method of claim 1 that comprises one or more of the following materials and components: neutral red powder; water for use in dissolving the neutral red powder; a filter or other absorbent material to which neutral red solution can or has been added to form a discrete area containing dried neutral red dye; a swab and/or bottle for collecting bodily fluids or other specimens from a subject; a mercury vapor or light emitting diode source of ultraviolet (UV) light; a UV light transmitting but visible light blocking filter, a light shielding compartment for placing; ethyl alcohol; pipettes, camera.
